(19) United States
(12) Patent Application Publication
(10) Pub. No.: US 2025/0018130 A1
(43) Pub. Date: Jan. 16, 2025

(54) INJECTION DEVICES

(71) Applicant: REPLICEL LIFE SCIENCES INC., Vancouver (CA)

(72) Inventors: Rolf Hoffmann, Freiburg (DE); Martin Hohlrieder, Feldkrich (AT)

(21) Appl. No.: 18/707,148

(22) PCT Filed: Nov. 11, 2022
(86) PCT No.: PCT/US2022/049736

§ 371 Date: May 2, 2024

Related U.S. Application Data
(60) Provisional application No. 63/278,209, filed on Nov. 11, 2021.

(51) Int. Cl. A61M 5/32; A61M 5/42; A61M 5/44

(52) U.S. Cl. CPC A61M 5/3298; A61M 5/422; A61M 5/44

(57) ABSTRACT

The present invention relates injection device for introducing a substance into tissue of a subject, the injection device comprising: (a) an application element (10); (b) a housing (50) having a receptacle for holding a syringe; (c) an electromechanical drive device system arranged to advance a piston for the syringe, and (d) a cooling assembly comprising a cooling plate (40), wherein the cooling plate (40) is incorporated into the application element (10). Moreover, the present invention also relates to a needle housing, liner and syringe specifically designed for fitting in the injection devices, according to the present invention or to be coupled thereto. Finally, the present invention relates to a method for introducing a substance into a tissue of a subject.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD OF THE INVENTION

The present invention relates generally to injection devices, and more specifically, to devices which are suitable for injecting, delivering or administering a variety of substances, including cells, into tissue. Moreover, the present invention relates to a needle housing and a liner specifically designed for fitting to the injection devices according to the present invention or to be coupled thereto. Finally, the present invention relates to a method for injecting, delivering or administering a variety of substances including cells, into tissue.

### BACKGROUND OF THE INVENTION

It is sometimes desirable to inject a substance into a subject with more precision than a typical syringe. In view of the presence of different layers, for example in a human being, it may be necessary to deliver a substance very precisely within just one distinct tissue layer. Different layers are distinguishable such as skin, mucosa, subcutis, fascia, muscle, nerves, or joints. For example, human skin comprises several different layers. The epidermis is the outer skin layer with a thickness of about 30 to 2000 m. The dermis is located beneath the epidermis with a thickness of about 500 to 1500 m. The subcutis is located beneath the dermis with a thickness of about 500 to 30,000 am. In certain applications, it is desirable to specifically deliver substances such as cells to specific locations within the skin.

There is therefore a need for means that allow that substances such as cells are injected in the desired physiological tissue. As one particular example, in the field of biological hair research there is a need for the accurate application of so called “dermal sheath cup” cells (“DSC”) which have potential for hair follicle regenerating. Similar demands exist concerning further injection applications. For example, the injection within the skin during cosmetic or aesthetic treatment requires further distinct means for the injection.

A further application concerns the injection into muscle tissue, joints, fascia, fat tissue, cartilage, submucosal tissues, or tendons. Such applications are often necessary after injuries which occur during sports or exercises. There is a constant need to treat tissue and distinct layers from the outside in a simple and reliable way.

The application of substances (e.g., liquids, biologics, or cell suspensions) within the dermis, epidermis or subcutis as well as muscle tissue layers or tendons requires a skillful handling by respectfully trained person. In addition, the application of such substances can involve distinct requirements. For example, with respect to cells there is a need to deliver them as near to the respective tissue layer which resembles the physiological tissue layer of these cells in situ as possible. Secondly, there is also a need to apply the cells in a very careful way. In particular cells, such as stem cells or freshly prepared primary cells, are sensitive in view of pressure which arises during the application. Such pressure occurs for example when cells are applied via a narrow cannula which results in a high compression of the cells. Such a compression and the resulting shear stress which acts on the cells are harmful. The consequence of this can be that cells are severely damaged and probably even lose their viability.

For a variety of applications which are not medically required (e.g., cosmetic procedures) it is necessary that the injection device can be used not only by a physician but also by cosmeticians or comparably trained persons. Therefore, there is also a need for injection devices ensuring a safe handling for such applications.

It is possible to apply cells to the scalp of a subject with a standard syringe having a needle of a distinct size and length. This has however the disadvantage that it is not possible to apply the cells in a constant and reproducible manner since the needle is shifted each time the injection site is changed. Accordingly, the angle between the injection needle and the scalp surface is not constant and will be very likely altered during each injection. Further, the injection depth will very likely vary during the different injections, since there is no measure of how deep the injection has to be performed if it is conducted with a standard syringe having an injection needle which is only guided by hand. In addition to the above, injection with needles can be a painful process, particularly when multiple injections (with one or more needles) is necessary.

A number of injection devices have been described in the art, including for example those disclosed in WO 94/23777, WO 02/083216, WO 2005/025641, WO2008/139303, WO2010/077596, WO2011/163382, WO 2013/113121, WO2013/156524 and WO 2016/022865. Notwithstanding the above, there is a need in the art to provide a device which overcomes the problems as described above and which allows the delivery of substances, in particular biological substances, such as cells, in a careful manner and at the desired physiological site in a safe, reliable, reproducible, painless and highly efficient way, wherein sterility of the injection device can be maintained without influencing the life time of the device. This underlying technical problem is solved with the injection device as defined within the claims.

For the purpose of illustration of the present invention, the present invention is shown in illustrative form, it being understood however, that the invention is not limited to the precise form shown in the figures or examples.

### SUMMARY OF THE INVENTION

Briefly stated, the present invention provides devices for injecting (e.g., administering, infusing, introducing or delivering) a variety of substances to a subject in a uniform and reproducible manner.

Within one aspect of the invention injection devices are provided for introducing a substance into a subject, comprising: (a) an application element (10); (b) a housing (50) having a receptacle for holding a syringe; (c) an electromechanical drive device system arranged to advance a piston for the syringe, and (d) a cooling assembly comprising a cooling plate (40), wherein the cooling plate (40) is incorporated into the application element (10). Within various embodiments of the above the cooling plate (40) is configured to cool down an upcoming injection site by contacting said injection site.

Within other embodiments the injection device has a drive system for advancing a piston of the syringe and a drive system for advancing the syringe itself. In one embodiment both drive systems may be advanced simultaneously, but the syringe may be retracted separately at a faster rate than the piston (e.g., the syringe may be retracted and the piston remain static, thereby injecting fluid into the subject). Within other embodiments the syringe and piston may be advanced simultaneously, followed by further advancement of the syringe (thereby injecting fluid into the subject). Within yet further embodiments the drive systems may be adjusted, for example, by the user or by a set protocol (e.g., to account for different viscosities of an injected substance).

Within certain embodiments of the invention a syringe (having an outer housing, a plunge and a rod attached to the plunger) can be loaded with a desired injectable substance. The syringe is then inserted into the injection device, and the rod removed (e.g., by unscrewing the rod from the plunger). A piston from the injection device can then be actuated in order to replace the rod, and upon command from a control unit drive the plunger forward.

Within yet other embodiments, the cooling assembly comprises a cooling shoe (45), a cooling element, in particular a Peltier element (41), a heat pipe (42), a heat exchanger (43) and at least one fan (44). Within various embodiments the housing (50) has intake vents (51) and outflow vents (52). Within certain embodiments of the invention the intake vents may be laterally arranged on the housing. Within other embodiments the housing (50) has outflow vents (52), which may be arranged at the back and/or the bottom of the proximal part of the injection device.

Injection devices of the present invention may optionally have one or more sensors, including for example, mechanical sensors, non-mechanical sensors (e.g., optical sensors; magnetic sensors and temperature sensors.

Within various aspects of the invention needle housings having one or more needles attached. Within certain embodiments of the invention, attachment of the needle housing to the injection device activates the injection device.

Within further aspects of the invention liners (e.g., caps, housing, sheets or similar components which can cover the surface of an application element) are provided. Within preferred embodiments the liners are non-pyrogenic and sterile. Within other embodiments at least a portion of the liner is comprised of a heat-conducting layer.

Within other aspects of the invention control units are provided which activate and control the injection device.

Also provided are methods for introducing a substance into the tissue of a subject comprising the steps of: a) selecting a first exterior surface of skin of a subject as a first injection site; b) placing the cooling plate of an injection device as described herein on a first injection site and cooling it to a desired temperature; c) moving the injection device in a position allowing an injection into the previously cooled injection site; d) penetrating the first surface with a needle or simultaneously more than one or all needles of a needle array of the injection device, wherein all needles of the needle array extending from the first surface by a uniform distance into the subject; e) ejecting a substance out of the needle/s and into the tissue of the patient; and f) withdrawing the one needle or simultaneously all needles of the needle array from the subject; g) optionally, simultaneously to step d), e) and/or f) cooling the upcoming injection site to a desired temperature; and h) optionally, repeating steps c) to f) and optionally g).

Within other embodiments of the invention the methods of the present invention can be performed by using an injection device according any of the embodiments as described herein.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### DESCRIPTION OF THE DRAWINGS

Features of the present invention, its nature and various advantages will be apparent from the accompanying drawings and the following detailed description of various embodiments.

FIG. 1 shows a perspective front/side view of an embodiment of the injection device (100) with a liner (11) attached to the application element (10), wherein the needles (20) are in their starting position. The injection device (100) comprises a housing (50) with intake vents (51) and a flap (53) which can be opened for inserting a syringe in the respective receptacle of the injection device. Moreover, the injection device comprises a communication interface (30) and a light source (60).

FIG. 2 shows a perspective back/side view of a preferred embodiment of the injection device with its intake vents (51) and outflow vents (52). The application element (10) is connected to the proximal part of the injection device by connection part (14).

FIG. 3 shows a longitudinal section of an embodiment of the injection device. The Peltier element (41) is coupled to the heat pipe (42) which transfers the heat from the Peltier element (41) to the heat exchanger (43). The heat exchanger (43) is located in the proximal part of the injection device below the electromechanical drive device system (71). The heat exchange of the heat from the heat exchanger (43) into the surrounding air is promoted by at least one fan (44) which is located in proximity to the heat exchanger (43). The power which is required for some of the parts of the injection device as e.g., the electromechanical drive device system, the Peltier element, the fan, the light source and the communication interface is provided over the data and power cord (70).

FIG. 4 shows in perspective front/side view the isolated cooling assembly of a preferred embodiment of the injection device except for any fans. The cooling plate (40) is part of the cooling shoe (45) which is mounted to the cold side of the Peltier element (41). The hot side of the Peltier element is mounted to the heat pipe (42) which transfers the heat of the Peltier element to the heat exchanger (43). The heat exchanger (43) comprises several parallel arranged cooling fins (47) on both sides of its extension of the heat pipe.

FIG. 5 shows a longitudinal section of the distal part of an embodiment of the injection device. Particularly, it shows a longitudinal section of the application element (10), the cooling shoe (45) with its cooling plate (40), the Peltier element (41), partly the heat pipe (42), the heat spreader (48), and the needle housing (21) in the retracted position with its needles (20). The mounting of the cooling shoe (45) in the housing of the injection device is shown which allows that air can pass by for avoiding or reducing condensation of moisture at the cooling shoe.

FIG. 6 shows in perspective front view the distal part of the injection device with its application element (10), the protruding portion (22) of the needle housing, the cooling plate (40) and a cut-out (46) in the cooling plate (40). The cut-out (46) may be a hole for a temperature sensor to permanently monitor the temperature of the cooling shoe. Moreover, the mounting of the cooling shoe (45) in the housing of the injection device is shown, which allows that air can pass by for avoiding condensation of moisture at the cooling shoe (45).

FIG. 7 shows a perspective front/side view of a preferred embodiment of the injection device (100), wherein the liner (11) is shown but not attached to the application element (10). The cooling plate (40) is incorporated in the application element (10). The injection device has a bulge (54) at its bottom of its rear center comprising the intake vents (51). The communication interface (30) is located in the housing (50) at its distal part between the position of the needle housing and the bulge (54).

FIGS. 8A, 8B, 8C, 8D and 8E show each an unattached liner (11) which can be attached to a preferred embodiment of the injection device. In FIG. 8A the complete distal part of the liner which consists of a heat conducting material (e.g., aluminum). In FIG. 8B only a rectangular area of the distal part of the liner consists of a heat conducting foil. Said rectangular area is located in the base area of the liner in that it covers the cooling plate after it has been attached to the application element. FIGS. 8C, 8D, and 8D show different perspectives of the liner.

FIG. 9 shows in perspective front/side view the distal part of the injection device with its application element (10) to which a liner (10) is attached. The liner (11) has the same form as the application element (10). Thus, both the application element (10) and the liner (11) have a base area with a width (12) and two flanks with a width (13). The means of lightning (60) are centrally arranged in the connection part (14) at the level of the nine needles (20) in their retracted position (starting position).

FIG. 10 shows an embodiment of the needle housing (21) according to the present invention with nine needles (20) and a protruding portion (22) for fitting into the injection device.

FIG. 11 shows in perspective front/side view a cross section through the connection part (14) with the cooling shoe (45), the Peltier element (41), the heat pipe (42) and the heat spreader (48). The cooling shoe comprises on its left and right side each a groove (49) and the housing comprising at its left and right side each a respective tongue (55) by which the cooling shoe is fixed in the housing. Apart from that, the cooling shoe (45) is floating in the housing so that air can pass by and condensation of moisture at the cooling shoe (45) is avoided.

FIGS. 12A, 12B and 12C depict representative embodiments of several disposable products, including: FIG. 12A, a representative liner; FIG. 12B, a representative needle housing and needle house cover; and FIG. 12C a representative syringe having a plunger attached.

### DETAILED DESCRIPTION OF THE INVENTION

As noted above, the present invention relates to the provision of an injection device which allows the precise and targeted delivering of a substance, such as for example, liquid or cells, in the tissue, preferably in dermal tissue layers, of a subject. It may be used for broad medical applications (including dermatological and/or cosmetic procedures) requiring injections of specific volumes of material at specific depths.

In order to further an understanding of the various embodiments herein, the following sections are provided which describe various embodiments: A. Injection Devices and Accessories; and B. Methods of Use.

### A. Injection Devices and Accessories

The injection device (100) of the present invention has a distal end (at the front where the needle and the application element are located) and a proximal end (at the back and thus at the opposite side of distal end). Within one aspect of the invention a dermal injection device is provided for introducing a substance into the tissue of a subject, the injection device comprises an application element, an injector arranged for mechanical communication with at least one needle, a receptacle for holding a syringe comprising at least one injectable substance through at least one needle, an electromechanical drive device system arranged to advance and retract at least one needle and to push forwards the piston of the syringe, a communication interface, a housing and a cooling assembly comprising a cooling plate, wherein the cooling plate is incorporated into the application element and the cooling plate is configured to cool down the upcoming injection site by contacting said injection site.

In one embodiment, the cooling assembly comprises a cooling element, in particular a Peltier element, a heat pipe, a heat exchanger and at least one fan. The cooling plate is preferably the distal part of the cooling element or in contact with said cooling element. Preferably, the cooling element comprises a Peltier element. More preferably, the cooling element comprises a Peltier element and a cooling shoe. Thus, in one embodiment the cooling assembly comprises a cooling shoe, a cooling element, in particular a Peltier element, a heat pipe, a heat exchanger and at least one fan, preferably two fans. In a further preferred embodiment, the cooling assembly additionally comprises a heat spreader which distributes the heat to a greater surface for decreasing the thermal resistance.

The Peltier element can be composed of a single piece of metal, or, laminated pieces of metal (e.g., that have been soldered and/or glued together).

In one embodiment the cooling plate is part of the cooling shoe and the cooling shoe is in contact with the cooling element, in particular the Peltier element. Preferably, the cooling shoe is in contact with the cold side of the Peltier element. In a further embodiment, the distal end of the cooling shoe forms the cooling plate. The cooling shoe is configured to provide and store the cold provided by the Peltier element to the cooling plate. Accordingly, the cooling shoe is configured to absorb heat from the skin/tissue, in particular from the injection site, of the person who is receiving the injection. Said heat absorption results in the cooling of the skin/tissue, in particular the injection site. The cooling shoe is preferably formed of highly heat-conducting material such as metals. Preferably the cooling shoe is formed of copper, aluminum, titanium, carbon steel, stainless steel or nickel alloys. The term “heat-conducting” as used herein refers to a material having a thermal conductivity of at least about 1, 2, 3, 4, 5, 6, 7, 8, 9 or 10 W/(m*K). Preferably, the term is used for any material having a thermal conductivity of about 1 to about 400 W/(m*K), or about 2 to about 400 W/(m*K), or about 3 to about 400 W/(m*K), or about 4 to about 400 W/(m*K), or about 5 to about 400 W/(m*K), or about 6 to about 400 W/(m*K), or about 7 to about 400 W/(m*K), or about 8 to about 400 W/(m*K), or about 9 to about 400 W/(m*K), or about 10 to about 400 W/(m*K), or about 50 to about 400 W/(m*K), or about 100 to about 400 W/(m*K), or about 150 to about 350 W/(m*K).

In the context of the present invention the terms “to be in contact to” or “to contact” is used to describe that two items (or materials) are in direct contact which each other. Preferably, said terms are also used to describe that two items are in indirect contact which each other. An indirect contact takes place if the contact of two items takes place over a third item (or material). In that case said first item is in direct contact with the third item and the third item is in direct contact with the second item. Said third item can be composed of several parts or layers. In particular, the terms “to be in contact to” or “to contact” is used to describe that two items are connected with each other so that they can transfer heat and cold, respectively, to each other either directly or through a third item. Preferably, said third item has a thermal conductivity of at least about 1, 2, 3, 4, 5, 6, 7, 8, 9 or 10 W/(m*K). Preferably, said third item has a thermal conductivity of about 1 to about 400 W/(m*K), or about 2 to about 400 W/(m*K), or about 3 to about 400 W/(m*K), or about 4 to about 400 W/(m*K), or about 5 to about 400 W/(m*K), or about 6 to about 400 W/(m*K), or about 7 to about 400 W/(m*K), or about 8 to about 400 W/(m*K), or about 9 to about 400 W/(m*K), or about 10 to about 400 W/(m*K), or about 50 to about 400 W/(m*K), or about 100 to about 400 W/(m*K), or about 150 to about 350 W/(m*K). Tissue or skin of a human or animal subject has typically a thermal conductivity of about 0.1 to about 0.5 W/(m*K) and thus below 1 W/(m*K). According to the present invention the terms “to be in contact to” or “to contact” does not refer to a contact of a first item (or material) with a second item (or material) by a third item (or material), wherein said third item (or material) is tissue or skin of the subject who is receiving the injection.

As described above the cooling plate is configured to cool down the upcoming injection site by contacting said injection site. According to the present invention said contact may be a direct contact of the cooling plate with the injection site or a contact through any kind of liner (e.g., a housing, cap, layer, or other material which separates two items) which is placed between the cooling plate and the injection site. Said liner is preferably configured to contact both the cooling plate and the injection site. It is preferably composed of heat-conducting material as defined above. The liner is preferably composed of a metal, a polymer or a plastic or any other material being able to transfer heat and cold, respectively, from the cooling plate to the injection site. More preferably, said liner is a metal foil such as a foil consisting or comprising copper, aluminum, titanium, or it is of carbon steel, stainless steel or nickel alloys or of other heat-conducting materials such as heat-conducting polymers or plastics.

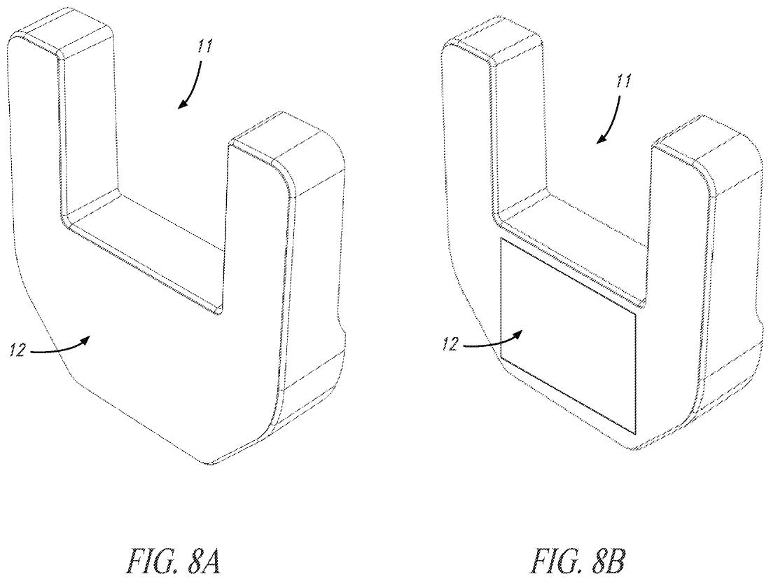

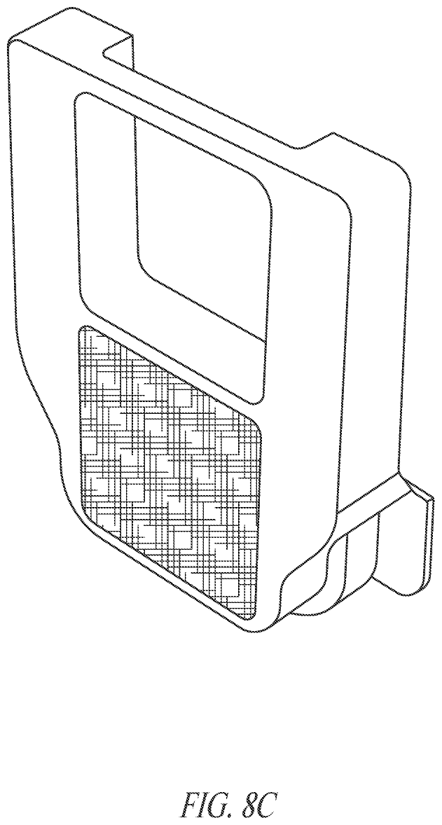

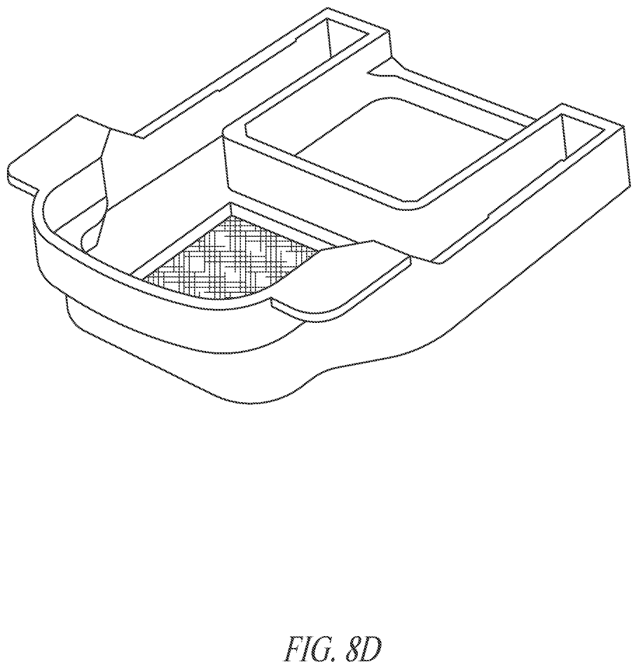

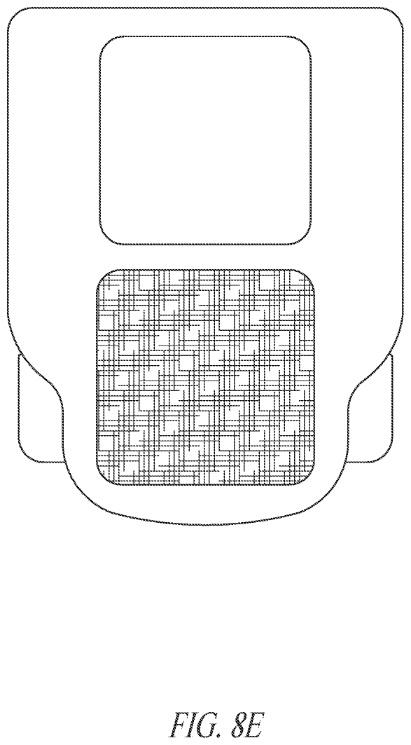

Within preferred embodiments the liner is sterile, and is used for retaining sterility of the cooling plate as described further below. Preferably, the liner has a thermal conductivity as described for the third item as defined above. The liner can also have a single-sided adhesive side, wherein the adhesive side of the liner is preferably the side which contacts the cooling plate. According to the present invention said liner is not the skin or the tissue of the subject who is receiving the injection. Within preferred embodiments of the invention the liner is approximately 2×2 cm in size (+/−0.5 cm). Representative examples of a liner are shown in FIGS. 8A-8E.

The hot side of the Peltier element is preferably in contact with a heat pipe. If the cooling assembly comprises additionally a heat spreader, the Peltier element is preferably in contact with the heat spreader and the heat spreader is in contact with the heat pipe. The heat pipe is configured for conducting the heat from the Peltier element and the heat spreader, respectively, to the heat exchanger. In the heat exchanger the heat is released, preferably into the surrounding area. The heat release is promoted by at least one fan. Preferably, the cooling assembly comprises one, two, three, four, five or six fans. In one embodiment the invention the injection device comprises two fans. The heat exchanger is preferably located at the proximal end of the injection device. Preferably, the heat pipe, the heat exchanger and/or at least one fan are located below the injector, cartridge and electromechanical drive of the injection device, preferably in a bulge of the housing. The heat exchanger is preferably located at the bottom proximal end of the injection device. In a preferred embodiment of the injection device according to the present invention at least one fan is located at the proximal end of the heat pipe in close proximity to the heat exchanger. In a further preferred embodiment, the cooling assembly comprises two fans which are located at opposite sides of the heat pipe at its proximal end and in close proximity to the heat exchanger. Preferably said two fans are laterally ranged, i.e., parallel to the left and right sides of the housing of the injection device.

The heat pipe, the heat spreader and the heat exchanger are made of heat-conducting material such as metals such as copper, aluminum, titanium, or of carbon steel, stainless steel or nickel alloys or of other heat-conducting materials such as heat-conducting polymers or plastics. They may be formed of the same or of distinct materials. Preferably, the heat pipe is configured to dissipate the heat from the cooling plate at the front (distal end) of the injection device to the heat exchanger which is preferably positioned at the back (proximal end) of the injection device. The heat exchanger is configured to receive the heat provided by the heat pipe. The heat exchanger is configured to have a very large surface. Within various embodiments it comprises several cooling fins for extending its surface. Preferably, the heat exchanger comprises 5 to about 200, more preferably about 10 to about 100 cooling fins. For increasing the heat transfer from the surface of the heat exchanger into the air the air flow over the surfaces of the heat exchanger is increased by at least one fan.

Within one embodiment, the distal surface of the cooling plate is configured for directly contacting the upcoming injection site. Said surface can have the form of a single area, preferably of a rectangle, a square, a circular area, or an oval area. Within certain embodiments, said surface does not have the form of a branched area and/or a composite area, which is composed of more than one area, such as two, three, four or more than four areas.

The upcoming injection site is the injection site into which the next injection is planned. Said next injection can be the first injection if no previous injection has been performed. The injection device is configured in that it has to be moved, preferably forwards or backwards, more preferably backwards, for allowing an injection into the previously cooled injection site. The injection device is further configured in that it cools down the next injection site while it is in the position for performing an injection in the previously cooled injection site. The injection device of the present invention may comprise one or more suitable mark(s) for facilitating that the injection is performed in the previously cooled injection site.

The distal surface of the cooling plate has preferably the same size as the injection site or a smaller or bigger size. Preferably, the part of the cooling element directly contacting the upcoming injection site has about 50%, about 60%, about 70%, about 80%, about 90%, about 100%, about 110%, about 120%, about 130%, about 140% or about 150% of the size of the injection site. In a preferred embodiment the size of the distal surface of the cooling plate is less than 2 cm². More particularly, the size of the cooling plate is preferably about 1 mm² to about 1.8 cm², more preferably about 5 mm² to about 1.6 cm², more preferably about 1 cm² to about 1.5 cm².

In a preferred embodiment, the distal part of the application element (10) is the part of the application element which contacts the skin of the subject who is receiving the injection. Said distal part comprises preferably a base area and two flanks which are arranged rectangularly to said base area. Accordingly, the distal part of the application element has preferably the form of a rectangular horseshoe or U. In other embodiments it has a horseshoe shape or a U-shape. Further shapes may be applicable for a skilled person. The application element is preferably connected with the remaining parts of the injection device by a connection part (14). Said connection part (14) is preferably covered by the housing and comprises the cooling element, preferably the Peltier element and the cooling shoe, and the beginning of the heat pipe. The connection part (14) is configured to be passed by at least one needle if at least one needle is advanced into the injection site. The arrangement of the application element provides an opening for at least one needle. Moreover, it facilitates that the distance of at least one needle and the skin of the subject who is receiving the injection can easily be kept constant. This in turn is important for exactly defining the injection depth of at least one needle. Moreover, the arrangement allows the operator of the injection device a good view of the injection site. The widest part of the base area of the distal part of the application element (12) is preferably at least 1.5 times as wide as the flanks (13). Preferably, it is about 1.5 to about 10 times as wide, or about 2 to about 5 times as wide. The cooling plate is preferably incorporated in the base area of the distal part of the application element.

Other embodiments of the present invention include injections devices, wherein the injection depth of the injection needle is in a range of about 0.5 mm to about 5 cm, or about 0.5 mm to about 4 cm, or about 0.5 mm to about 3 cm, or about 0.5 mm to about 2 cm, or about 0.5 mm to about 1 cm, or about 0.5 mm to about 5.5 mm, or about 1.5 mm to about 4.0 mm, or about 2.0 mm to about 3.5 mm, most preferably about 3.4 mm. In an especially preferred embodiment the injection depth of the injection needle is about 0.5 mm to about 2.5 cm, more preferably of about 1 mm to about 23 mm.

The injection device of the present invention is preferably configured to allow several injections per minute. Preferably, it is configured to allow 3, 4, 5, 6, 7, 8, 9, 10, 11, 12 or more injections per minute.

The cooling assembly is preferably configured to cool down the injection site to a temperature below about 8° C., preferably to temperature of about 1° C. to about 8° C., or about 1° C. to about 7° C. In an especially preferred embodiment the target temperature range is of about 1° C. to about 4° C.

In various embodiments the cooling element, for example a Peltier element, has a peak power of about 15 W to about 290 W, more preferably of about 15 W to about 100 W, more preferably of about 20 W to about 50 W, more preferably of about 30 W to about 50 W. In a further preferred embodiment, the cooling element, preferably the Peltier element, has a peak power of about 10 W to about 50 W. The peak power of the Peltier element is the maximum amount of power usage of the Peltier element.

In a further embodiments the cooling element, for example a Peltier element, has a total usable cooling power of at least about 3 W. Preferably, the cooling element has a cooling power of about 3 W to about 30 W, or about 3 W to about 20 W or about 3 W to about 10 W or about 4 W to about 30 W or about 4 W to about 20 W, or about 4 W to about 10 W. The total usable cooling power or cooling power of a Peltier element is the power which is transported by the Peltier element as heat and cold, respectively. In particular, it is the power with which heat is absorbed by the Peltier element.

In yet other embodiments the cooling power per area of the cooling plate is about 1.5 W/cm² to about 15 W/cm². More preferably it is about 2 W/cm² to about 10 W/cm² or about 3 W/cm² to about 8 W/cm². Preferably, the cooling power per area provided to the injection site is about 1.5 W/cm² to about 15 W/cm². More preferably it is about 2 W/cm² to about 8 W/cm² or about 3 W/cm² to about 7 W/cm², most preferably about 4 W/cm² to about 6 W/cm².

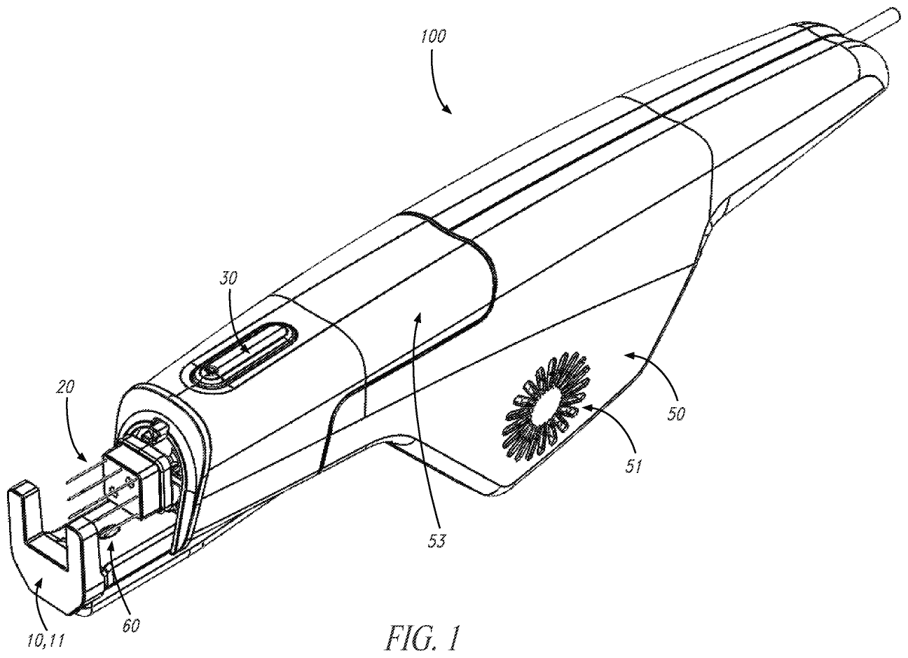

The housing of the injection device according to the present invention covers preferably most parts of the injection device as e.g. the injector, the receptacle, the electromechanical drive device and the parts of the cooling assembly except for the cooling plate. In particular, the housing covers preferably the heat exchanger, at least one fan, the heat pipe and the cooling element, in particular the Peltier element. The communication interface is preferably incorporated into the housing and operable at the surface of the housing. Preferably, the communication interface is located at the top of the injection device as e.g. shown in FIGS. 1, 7 and 9. In a preferred embodiment, the application element is part of the housing. At least one needle is preferably not covered by the housing. The needle housing may be partly covered by the housing.

In a further embodiment, the housing is configured to minimize the surface of the cooling element, in particular the Peltier element, and/or the cooling shoe which contacts the housing. This results in that air can easily flow past the cooling element, in particular the cooling shoe. Accordingly, condensation of moisture at the cooling element, in particular the cooling shoe, is avoided or minimized. Accordingly, the cooling element, in particular the cooling shoe, are mounted only at certain positions to the housing of the injection device. Preferably, they are mounted in the housing in as few places as possible which are necessary for fixing them in the housing. For example, the cooling shoe is mounted on single elevations in the housing such as on ribs, fins or protruding portions of the housing. Alternatively or in addition, the cooling element, in particular the cooling shoe, may be fixed by a tongue-and-groove joint in the housing. For examples, the cooling shoe may comprise a groove and the housing may comprise a respective tongue fitting into the groove of the cooling shoe or the housing may comprise a groove and the cooling shoe may comprise a respective tongue fitting into the groove of the housing. Apart from that and optional some additional contact or fixing positions in the housing, the cooling element, in particular the cooling shoe, is preferably floating in the housing so that air can pass by and condensation of moisture at the cooling shoe is avoided.

Alternatively or additionally, the cooling element, in particular the cooling shoe, and/or the housing may comprise cut-outs that increase the surface air can flow past. In a preferred embodiment, the cut-out (46) may be a hole for a temperature sensor to permanently monitor the temperature of the cooling shoe. Thus, in a preferred embodiment of the present invention the cooling shoe is embedded in the injection device in that about 50 to about 99%, about 60 to about 99%, about 70 to about 99%, or about 80 to about 99% of its surface is surrounded by air. In a further preferred embodiment, the cooling shoe is mounted in the housing by means in that about 1 to about 50%, 1 to about 40%, 1 to about 30%, or 1 to about 20% of each of their surfaces are in contact with the housing.

In a further embodiments, the housing of the injection device has intake vents, preferably laterally arranged, and outflow vents, preferably at the back and/or the bottom of the proximal part of the injection device. Preferably, the housing comprises at least one intake vent through which air is taken in by at least one fan and at least one outflow vent through which air is blown off by at least one fan. Preferably, the housing of the injection device comprises 2 to 50, 5 to 40 or 10 to 40 intake vents and 2 to 50, 5 to 40 or 10 to 40 outflow vents. Preferably, the intake and outflow vents are positioned at the housing of the injection device in order to direct the airflow away from the subject and the operator of the injection device, in particular the upper parts of the operator such as the face and the hands. Accordingly, the intake vent/s is/are preferably positioned at one or both sides of the injection device in particular at the bottom of said one or both sides. The outflow vent/s is/are preferably positioned at the back (proximal end) of the injection device. Optionally, said outflow vent/s is/are positioned at the bottom side of the injection device and/or at the side facing away from the front (distal end) of the injection device. Preferably, the intake vent/s is/are positioned in close proximity to the fan/s. The outflow vent/s is/are preferably positioned in close proximity to the heat exchanger. In a preferred embodiment the housing forms a bulge at its bottom in which the heat pipe, at least one fan and the heat exchanger are located. If the housing comprises such a bulge, the intake and outflow vents are preferably located in said bulge.

In other embodiments of the invention, the injection device of the present invention comprises as least one non-mechanical sensor such as an optical sensor, a magnetic sensor or another non-mechanical sensor. Non-mechanical sensors such as optical sensors and magnetic sensors have generally a longer lifetime than mechanical switches, in particular for miniaturized switches. Moreover, the useful life of the injection device can be quantified because the number of uses of non-mechanical sensors such as optical sensors and magnetic sensors is known. For example, the injection device may comprise a non-mechanical sensor arranged to pass information associated with a position of at least one needle. Further examples for non-mechanical sensors which may be incorporated into the injection device according to the present invention are
a sensor arranged to pass information associated with the receptacle or the cartridge,
a sensor which detects the temperature at the cooling plate or the cooling shoe,
a sensor which detects the presence of a syringe inserted into the receptacle or cartridge
a sensor which detects an emptied syringe.
a sensor that indicates that the lid or flap of the syringe compartment has been closed
environment sensors configured to produce environment data samples. For example, said environment sensors may sense temperature, humidity, altitude, air density, air pressure, ambient light, motion, or other environmental conditions. The environmental data may be cooperatively used with other data by the processor to calculate ambient background light, temperature of the cooling plate, and many other parameters.

In one embodiment of the injection device according to the present invention, the injection device comprises 1, 2, 3, 4, 5, 6, 7, 8, 9 or 10 non-mechanical sensors or 1 to 20 or 1 to 30 non-mechanical sensors. Preferably, it comprises the following non-mechanical sensors: one, two, three or more NTC (Negative Temperature Coefficient) thermistor(s); one, two, three or more magnetic sensor(s); one, two, three, four, five, six, seven, eight or more current sensor(s); and/or one, two, three or more photoelectric sensor(s). The control unit may also comprise non-mechanical sensors such as one, two, three, four, five, six, seven, eight, nine, ten or more capacitive touch sensors. The injection device may also comprise 1, 2, 3, 4, 5, 6, 7, 8, 9 or 10 mechanical sensors or 1 to 20 or 1 to 30 mechanical sensors. Preferably, it comprises one, two, three, four or more mechanical switches. In a preferred embodiment the injection device comprises a mechanical switch at the injection device for starting the injection and one mechanical switch at the control unit for turning the device on and off. In a preferred embodiment of the present invention, the injection device comprises a temperature sensor in a hole of the cooling shoe and/or the cooling plate to permanently monitor the temperature of the cooling shoe and/or the cooling plate.

In other embodiments of the invention, the injection device according to the present invention has a needle housing comprising at least one needle. Said needle housing is preferably sterile and disposable. The needle housing may contain an array of needles, and may be referred to as a pincushion design. In various embodiments, the array of needles contains 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 needles. In other embodiments, the array of needles may contain 9 to 100 needles. Within further embodiments the needles may have a gauge of 18, 19, 20, 21, 22, 23, 24, 25, 26, or 27. Especially preferred are a plurality of needles such as 4, 9, 16, 25 or 36. The array of needles is advantageous in that it allows for the injection of large volumes of material at a single time and/or at several distinct injection points.

Within various embodiments of the invention the needle size or gauge may be anywhere from a 10 to a 34 gauge needle (where the higher the gauge of the needle represents a smaller needle size), although smaller needle sizes (e.g., greater than 22 gauge, such as 23, 24, 25, 26, 27, 28, 29, 30, 31, 32 or 33 gauge) are generally preferred. Within certain embodiments of the invention, the needle housing may comprise more than one needle size (e.g., several needles with one size, and other needles with a different size).

Within yet another embodiment all injection needles are configured to penetrate simultaneously into the body of the subject. Preferably, the injection device is configured in that all needles of the needle array extend by a uniform distance into the tissue of the subject. Several distinct injection points into which injections are performed simultaneously in one injection step by a needle housing comprising more than one needle may be considered as one injection site. If the injections device comprises only one needle, the injection site is the site of the tissue of the subject who is receiving the injection into which the injection is performed with said one needle.

In various embodiments the needle housing comprises means configured to fit particularly into the injection device according to the present invention. For example, the needle housing may comprise one or more protrusions or projections which fit into and preferably, couple with the injection device. In another embodiment of the present invention the injection device comprises a protrusion or projection which fits into and preferably couples with a respective cut-out in the needle housing. In a further preferred embodiment, the injection device or part thereof is configured to be activated only if a needle housing is coupled to the injection device. For example, the injection device or a part thereof, as e.g. the electromechanical drive device, is only activated or operable after a needle housing comprising said one or more protrusions or projections has/have been inserted into the injection device. In a further preferred embodiment, the needle housing and the injection device comprise means for locking the needle housing into the injection device for allowing a precise and safe injection. Accordingly, the needle housing and the injection device may comprise a matching locking mechanism so that the needle housing is firmly coupled to the injection device and is only released after the locking mechanism is released. The means for activating the injection device and locking the needle housing into the injection device may be the same or they may be distinct ones.

The application element and the cooling element are preferably incorporated into the injection device according to the present invention. That means that they are optionally not configured to be easily removable from the injection device e.g. for being sterilized after each use. Accordingly, the application element is configured to retain sterility. Sterility of the injection device is preferably retained by a liner, housing or cap, which is configured to be easily put on and off the application element. Thus, in a preferred embodiment of the injection device according to the present invention, the application element is configured to be coupled to a liner (11), housing or cap. Said liner, housing or cap is preferably, apart from the injection needle/s, the only part of the injection device which comes into contact with the subject who is receiving the injection for sterility reasons. Thus, said liner, housing or cap is preferably configured to be put on before the injection device is used for any new injection or any new subject who is receiving the injection. Preferably said liner, housing or cap can be sterilized and is configured for repeated use after sterilization. Alternatively, said liner, housing or cap is sterile and disposable. The liner, housing or cap has preferably the same form as the application element. In particular, the distal part of the liner, housing, or cap which contacts the skin of the subject who is receiving the injection has preferably the same form as the distal part of the application element.

The communication interface of the injection device according to the present invention can comprise a button or trigger which initiates the injection. This button is pressed after the injection device has been brought into the proper position.

The injection device according to the present invention comprises a receptacle for holding a syringe which contains the substance to be injected. The receptacle is preferably enclosed by the housing of the injection device. It is preferably movable so that an inserted syringe for holding the substance to be injected can be advanced and retracted together with at least one needle. The housing preferably also encloses the injector which is preferably also movable. The injector is preferably configured to be coupled to at least one needle or a needle housing comprising said at least one needle at the distal end of the injector. At the proximal end of the injector the injector is attached to the receptacle for a syringe. Accordingly, the injector is preferably movable together with at least one needle and the receptacle and the syringe, respectively, so that it can be advanced and retracted by the electromechanical drive device.

In preparation for operation, a syringe can be placed in the receptacle. The receptacle may be an enclosed space, accessed through a lid or flap (53) that is removable and can be fitted back in place after the syringe has been removed or placed in the injection device. In a preferred embodiment, the lid may be attached to the injection device via a hinge which allows the lid to be opened and closed. In a further preferred embodiment, the injection device comprises a cartridge arranged to hold at least one injectable substance through at least one needle (20). In still a further preferred embodiment, the injection device contains a syringe. The syringe is preferably disposable and sterile.

Within various embodiments the injection device contains a motor assembly comprising an electromechanical drive device system for advancing and retracting at least one needle and for pushing forward the piston of the syringe for injecting the substance via at least one needle. The electromechanical drive device system may comprise one or more drive motors. It may comprise a delivery mechanism including a motor whereby at least one needle is moved from a retracted (protected, non-exposed, starting) position to an extended position where it can be used to inject a substance into a subject. Independent from the movement of at least one needle, the volume of the syringe may be adjusted to expel the contents thereof through at least one needle and into a subject in need thereof. The injection of the substance is conducted with a motor which allows that the piston of the syringe is pushed forward thereby injecting the liquid via at least one needle. Optionally, the injection device of the present invention is configured to inject the substance during the retraction movement of at least one needle. Alternatively, it may be configured to inject the substance during the forward movement or after the forward movement and before the retraction movement, i.e. if at least one needle has reached the desired injection depth. Preferably, the injection device is configured in that different injection volumes are injectable at different injection depths. Examples of motor assemblies, electromechanical drive device systems and methods for controlling the same can also be found, for example, in WO 2016/022865 which is incorporated by reference in its entirety.

Optionally, the injection device further comprises a light source (60). Such lighting may be, but is not restricted to, LED (light-emitting diode) or other lighting appliances. The provision of lighting appliances allows a better visualization and an easier determination of the distinct injection site. In other embodiments of the present invention, the light source is located at the distal end of the injection device (e.g., in the connection part (14)) in order to illuminate the injection site. The light source (60) can also be covered by the housing of the injection device, wherein a cut-out in the housing allows the emitted light to light up the injection site. Said cut-out may be covered by a transparent layer or material.

Preferably, the injection device includes a comfortable handle. Optionally, it also comprises a connection to a data cord and/or a power cord (70). Preferably, the data cord is also the power cord. Alternatively, the injection device might be operable by a power source incorporated into the injection device as e.g. by a disposable or rechargeable battery. The injection device is preferably light and well balanced in order to make is easy to handle.

In a further embodiment, the injection device may be communicatively connectable to a control unit (e.g., a mobile device such as a laptop, pad, or cell phone, a desktop device, or a device designed solely for use with the injection device). Preferably, the injection device is communicatively connectable to a control unit through a communication interface on the injection device. The connection can be a cable connection, e.g. via the data cord, or a cable free connection such as via Bluetooth or WLAN. In a further preferred embodiment, information gathered e.g. from the sensors of the injection device are provided to the control unit. For example, the cooling element is preferably arranged to provide temperature information to a control unit. The control unit comprises preferably a processor and a memory arranged to store processor executable instructions.

The present invention also refers to a needle housing which is configured for fitting into the injection device according to the present invention, wherein the needle housing is locked in the injection device after its insertion. Said needle housing comprises a protrusion or projection which fits into the injection device. Alternatively or additionally, the needle housing may comprise a cut-out into which a protrusion or projection of the injection device fits. The needle housing according to the present invention comprises the features as described above. In a preferred embodiment of the present invention, the fitting of the needle housing and the injection device is required for activating the injection device according to the present invention or a part thereof such as the electromechanical drive device.

The present invention also refers to a liner (e.g., a housing, cap, layer, or other material which separates two items) which is configured to be coupled to the application element of the injection device according to the present invention. Said liner, housing or cap comprises a heat-conducting liner, part or item which contacts the distal surface of the cooling plate after its coupling to the application element. Said heat-conducting liner, part or item is configured to transfer heat and cold, respectively, from the cooling plate to the skin of a subject who is receiving an injection. In particular, it is configured to transfer heat and cold, respectively, from the cooling plate to the upcoming injection site, preferably with a cooling power per area of about 1.5 W/cm² to about 10 W/cm², more preferably of about 2 W/cm² to about 10 W/cm². The liner contacting the cooling plate is preferably composed of copper, aluminum, titanium, or it is of carbon steel, stainless steel or nickel alloys or of other heat-conducting materials such as heat-conducting polymers or plastics. More preferably, said liner is a metal foil such as a foil consisting or comprising copper, aluminum, titanium, or it is a foil of carbon steel, stainless steel or nickel alloys or of other heat-conducting materials such as heat-conducting polymers or plastics. It has preferably a thermal conductivity of at least about 1, 2, 3, 4, 5, 6, 7, 8, 9 or 10 W/(m*K). Preferably, it has a thermal conductivity of about 1 to about 400 W/(m*K), or about 2 to about 400 W/(m*K), or about 3 to about 400 W/(m*K), or about 4 to about 400 W/(m*K), or about 5 to about 400 W/(m*K), or about 6 to about 400 W/(m*K), or about 7 to about 400 W/(m*K), or about 8 to about 400 W/(m*K), or about 9 to about 400 W/(m*K), or about 10 to about 400 W/(m*K), or about 50 to about 400 W/(m*K), or about 100 to about 400 W/(m*K), or about 150 to about 350 W/(m*K).

The liner configured to contact the distal surface of the cooling plate may have the same size as the distal surface of the cooling plate. Alternatively, it may have a bigger size as the distal surface of the cooling plate. In a preferred embodiment the complete distal surface of the liner is composed of said heat-conducting material. The liner can be configured to contact the distal surface of the cooling plate and may have a single-sided adhesive side, wherein the adhesive side of the liner is preferably the side which contacts the cooling plate after the liner has been attached to the application element of the injection device. B. Methods of Use

The present invention also provides various methods of using an injection device according to the present invention. Thus, in one embodiment, the present disclosure provides a method for introducing a substance into tissue of a subject, the method comprising placing the substance into an injection device as disclosed herein and then injecting the substance into the tissue. The injectors of the present invention provide an improved level of control, precision and efficiency for injecting a substance or material into specific tissue, e.g., into epithelium (epidermis), intradermal (dermis), subcutaneous (fat), intramuscular tissue or into muscles. Within certain embodiments of the invention the injection devices provided herein may be used to inject into hard or thicker tissues, such as the palms and soles of the feet, scar tissue, cancers (e.g., of the epithelium) and hyperproliferative diseases of the skin.

For example, in one embodiment the present disclosure provides a method for introducing a substance into tissue of a subject comprising:
a) selecting a first exterior surface of skin of a subject as a first injection site;
b) placing the cooling plate of an injection device on the first injection site and cooling it to a desired temperature;
c) moving the injection device in a position allowing an injection into the previously cooled injection site;
d) penetrating the first surface with a needle or simultaneously with all needles of a needle array of the injection device, wherein all needles of the needle array extending from the first surface by a uniform distance into the subject;
e) ejecting a substance out of the needle/s and into the tissue of the patient; and
f) withdrawing the one needle or simultaneously all needles of the needle array from the subject;
g) optionally, simultaneously to step d), e) and/or f) cooling the upcoming injection site to a desired temperature, and
h) optionally, repeating steps c) to f) and optionally g).

In a preferred embodiment of the present invention said method is performed by using an injection device according to the present invention. In a further preferred embodiment step f) is performed while the one needle or simultaneously all needles of the needle array are withdrawn from the subject.

In a further preferred embodiment of the present invention the injection depth of the injection needle is in a range of about 0.5 mm to about 5 cm, or about 0.5 mm to about 4 cm, or about 0.5 mm to about 3 cm, or about 0.5 mm to about 2 cm, or about 0.5 mm to about 1 cm, or about 0.5 mm to about 5.5 mm, or about 1.5 mm to about 4.0 mm, or about 2.0 mm to about 3.5 mm, most preferably about 3.4 mm. In a further preferred embodiment, the substance is ejected into the dermis, epidermis, subcutis, muscle tissue layers or tendons of a human or animal subject.

In a further preferred embodiment said method is a non-therapeutic and non-surgical method (e.g., for cosmetic or aesthetic applications). Said non-medical applications do not result in a substantial physical intervention on the human or animal body of any subject and does not require professional medical expertise of a physician. Such non-medical applications can e.g. be performed by a cosmetician or a similarly trained person. Preferably, said non-medical applications does not comprise any treatment which entail a substantial health risk even when carried out with the required professional care and expertise. In a preferred embodiment the injection device of the present invention is particularly safe in application since it is configured for injections into human tissue through one or more layers of the dermis or epidermis, only. Said configuration can for example be performed by limiting the possible injection depth of at least one needle. In a further preferred embodiment, the non-therapeutic and non-surgical method according to the present invention is restricted to a method, wherein the substance is ejected into the dermis, epidermis or subcutis of a human or animal subject. The substance to be injected is a substance not entailing any substantial health risks or resulting in a substantial physical intervention on the human or animal body or it is injected in a concentration not entailing any substantial health risks or resulting in a substantial physical intervention on the human or animal body.

A wide variety of substances or injectable compositions can be administered to a subject utilizing the injection devices provided herein, including injectable aesthetic or cosmetic products, and injectable pharmaceutical products. Representative examples include, but are not limited to: fats (e.g., autologous fat), polymers (e.g., poly-L-lactic acid or “PLA” and polymethylmethacrylate or “PMMA”); calcium hydroxylapatite; pharmaceutical products (e.g. botulinim toxin, hyaluronic acid, collagen, antibiotics and steroids) and cells and cell products (e.g. Platelet Rich Plasma or “PRP) and cells (e.g., autologous mesenchymal stem cells or autologous dermal sheath cup cells).

For the purpose of illustration of the present invention, the present invention is shown in illustrative form, it being understood however, that the invention is not limited to the precise form shown in the figures or examples.

### EXAMPLES

This description of embodiments of the present invention is exemplary in nature and is in no way intended to limit the invention or its application or uses.

### Example 1

### Injection Device

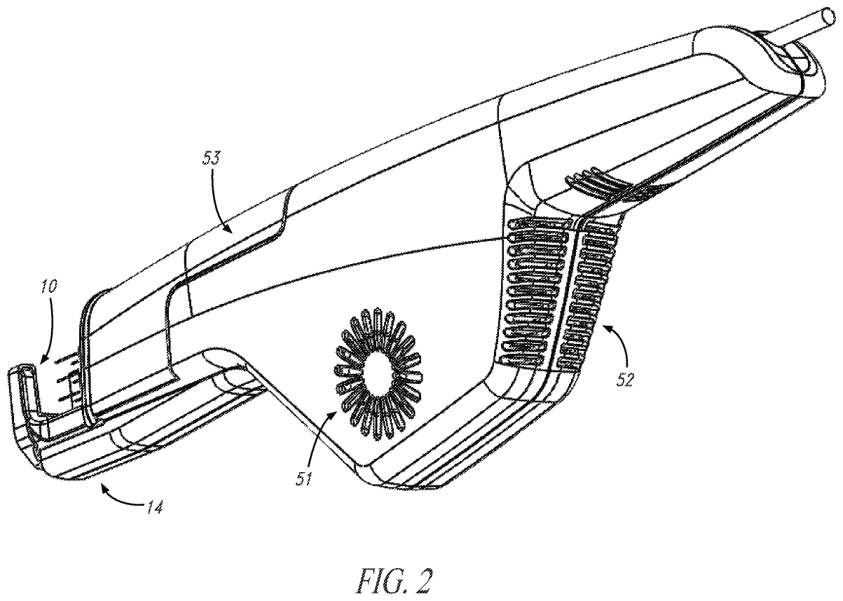

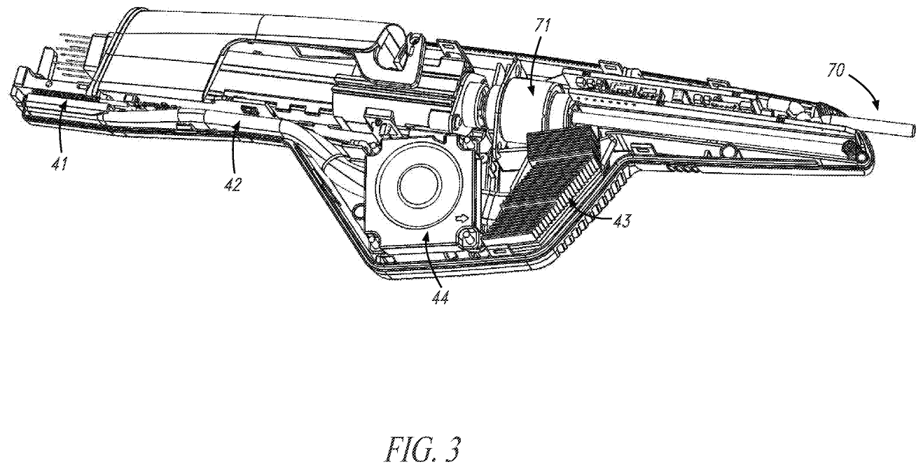

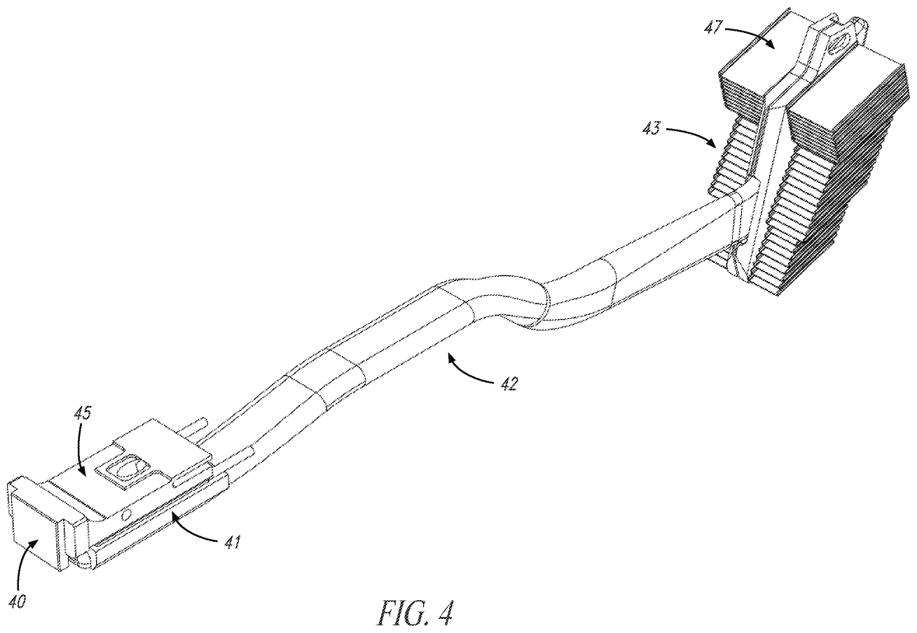

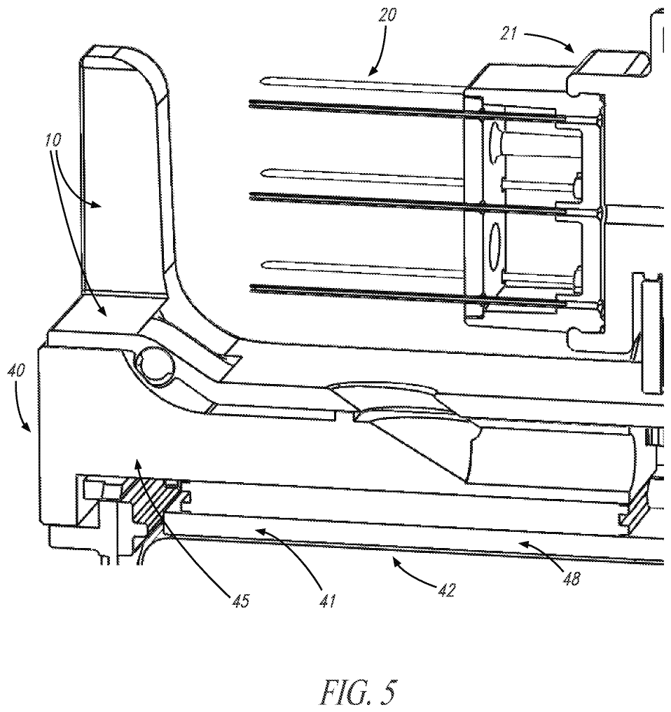

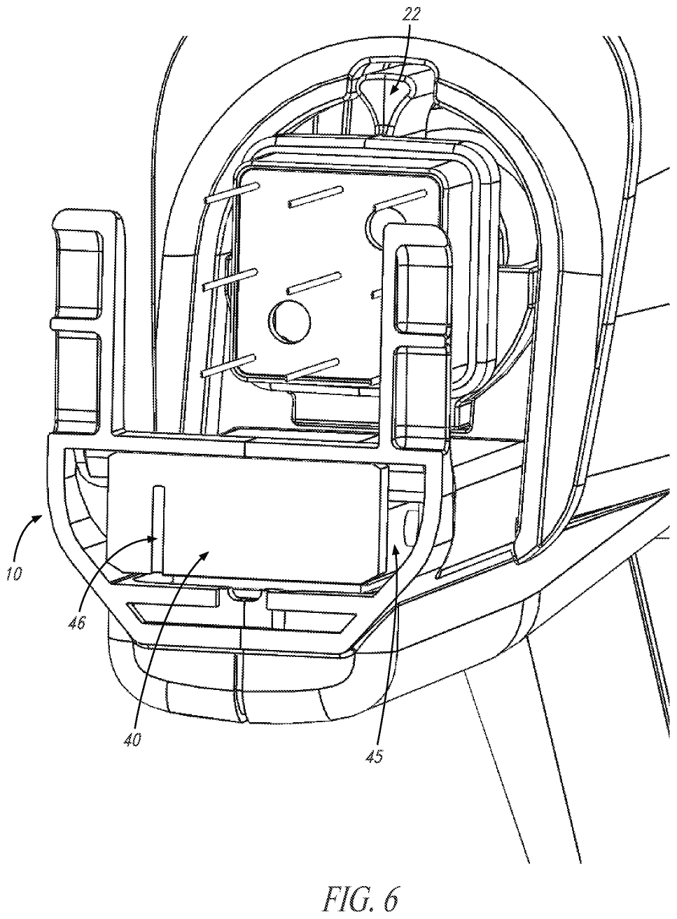

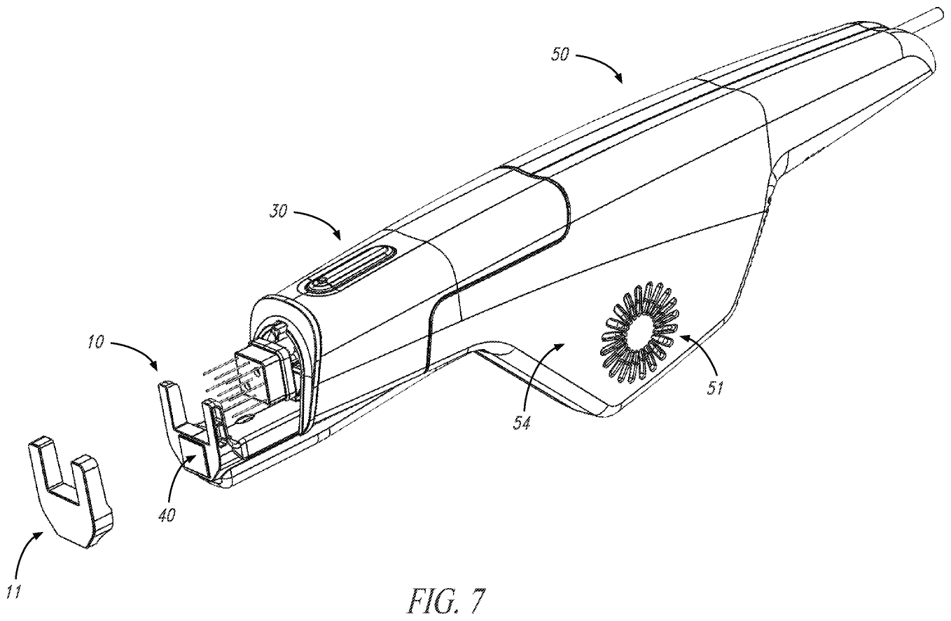

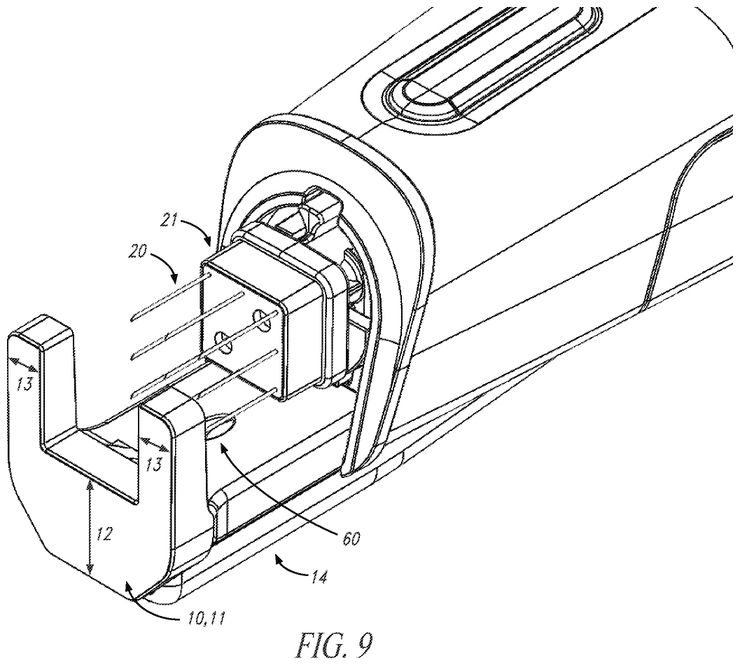

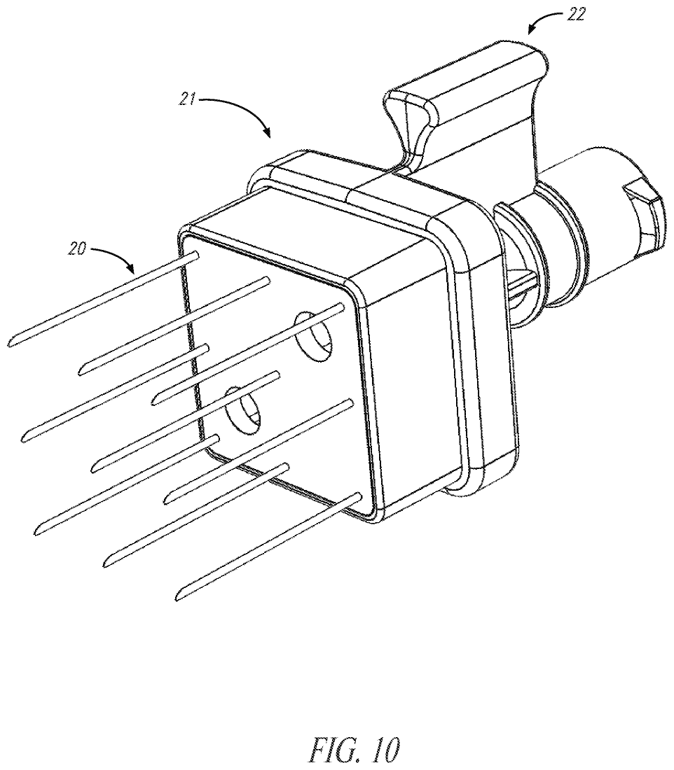

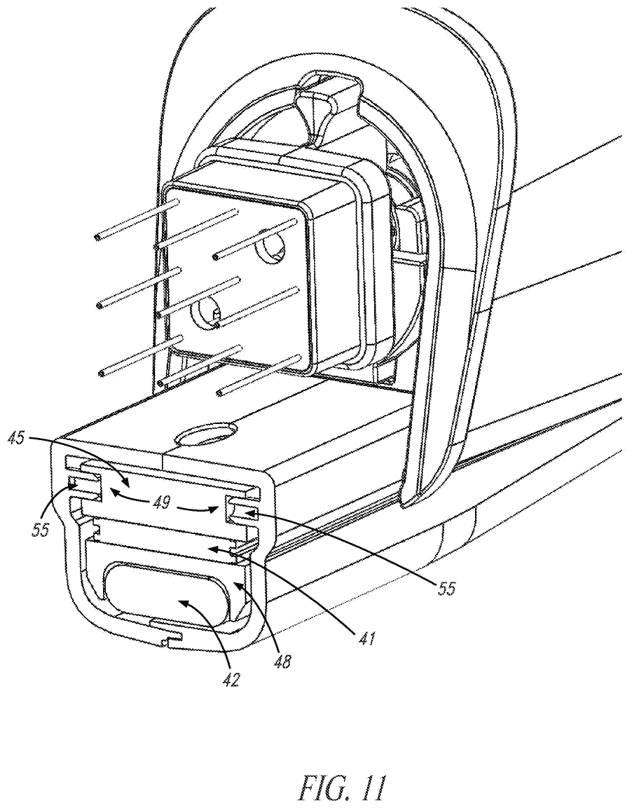

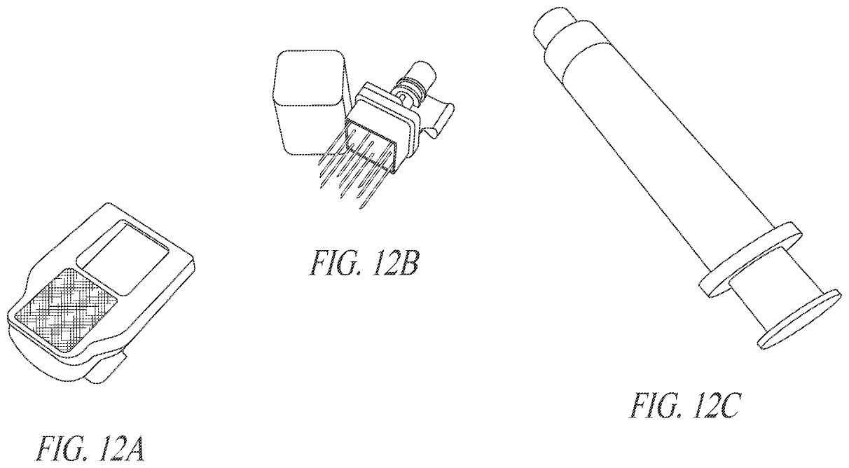

One embodiment of the invention an injection device is shown in perspective front/side view in FIGS. 1 and 7, in perspective back/side view in FIG. 2 and in longitudinal section in FIG. 3. FIG. 4 shows a preferred cooling assembly for the injection device except for any fans. FIG. 5 shows in longitudinal section the application element, the connection part and the needle housing. FIG. 6 shows in perspective front view the distal part of the application element comprising the cooling plate and the cooling shoe together with the needle housing coupled into the injection device. FIGS. 8A and 8B show in perspective front view preferred embodiments of the liner according to the present invention. FIG. 9 shows in perspective front/side view the distal part of the injection device with attached liner and coupled needle housing. FIG. 10 shows in perspective front/side view a preferred embodiment of the needle housing according to the present invention. FIG. 11 shows in perspective front/side view a cross section through the connection part (14). In FIGS. 1, 2, 3, 5, 6, 7, 9 and 11 the needle housing is shown in its retracted position.

As can be seen from the figures, the injection device comprises a head section as application element (10) for the application on the body of a subject, for example the skin of a subject. The application element (10) comprises a base area and two flanks which form a rectangular opening through which the needle housing (21) can advance to the injection site of a subject who is receiving an injection. The distal part of the application element (10) is directed to the body of the subject and comprises a cooling plate (40). The application element (10) is connected to the remaining part of the injection device by a connection part (14). The connection part (14) is configured in that the needle of the needle housing can advance to the injection site of the subject who is receiving the injection through the opening formed by the two flanks of the application element. The connection part (14) comprises the cooling shoe (45), the Peltier element (41) and the head part of the heat pipe (42) and is covered by a plastic housing (50). It also comprises LED as means for lightning the injection site. An injection needle housing (21) is coupled to the head section of the injection device by having a protruding portion (22) which fits into the housing of the injection device. The needle housing (21) comprises 9 needles (20). In the starting position the injection needles (20) are not covered by the housing (50). This allows the control of the application of the injection needle. The injection device comprises a cooling assembly consisting of a cooling shoe (45), a Peltier element (41), a heat spreader (48), a heat pipe (42), a heat exchanger (43) and two fans (44). The Peltier element has a peak power of about 33 W and a total usable cooling power of about 4.5 W. The cooling power provided at the cooling plate is about 3.5 W/cm². The heat production of the Peltier element (41) which is dissipated over the heat exchanger (43) is about 15 W during operation of the injection device. The cooling shoe (45) is composed of copper, wherein the distal end of the cooling shoe which contacts the injection site of the subject who is receiving the injection is the cooling plate (40). The cooling shoe (45) comprises a cut-out (46) which is a hole for a temperature sensor to permanently monitor the temperature of the cooling shoe. For avoiding condensation of moisture on the cooling shoe (45), the cooling shoe (45) is float-mounted in the housing (50) apart from a tongue-and-groove joint (49, 55) at its left and right side which fix the cooling shoe in the housing (50). The cooling plate (40) has the form of a square and an area of 1.2 cm². The cooling assembly (except for the cooling plate) is covered by the housing (50). The cooling assembly is arranged at the bottom side of the injection device below the needle housing (21), the injector, the receptacle for holding a syringe, and the electromechanical drive device system (71). The injection device has a bulge (54) at its bottom of its rear centre in which the two fans (44) and the heat exchanger (43) are located. The fans (44) are placed on opposite sides of the heat pipe (42) in close proximity to the heat exchanger (43). The heat exchanger comprises 30 cooling fins (47) on both (opposite) sides of its extension of the heat pipe. The housing of the injection device has 20 intake vents, which are laterally arranged at both sides of the bulge (54) in close proximity to the fan. The intake vents are designed as slots which are arranged in a circle. The housing (50) has eleven outflow vents at the back of the bulge (54) and four outflow vents at the bottom of the proximal part of the injection device. The outflow vents are positioned in close proximity to the heat exchanger and are designed as slots in a parallel arrangement. The housing has a flap (53) which can be opened for inserting the syringe in the receptacle in the injection device. The communication interface (30) is incorporated in the flap (53). The injection device is connected by a data and power cord (70) to a control unit (not shown). The injection device comprises the following sensors: two NTCs, one magnetic sensor, nine capacitive touch sensors at the control unit, six current sensors and one photoelectric sensor. Moreover, it comprises one mechanical switch at the injection device for starting the injection and one mechanical switch at the control unit for turning the injection device on and off. In FIGS. 1 and 9 a sterile liner (11) is attached to the application element of the injection device and in FIG. 7 the application element (10) and the liner (11) are shown separately. FIG. 8A shows the liner of FIGS. 1, 7 and 9 in more details. Its whole distal part which contacts the subject who is receiving the injection is composed of a single-sided adhesive aluminum foil. The remaining parts of the liner are made of plastic. The adhesive side of the aluminum foils is the proximal side of the aluminum foil which faces away from the subject who is receiving the injection and towards the cooling plate if the liner is attached to the application element (10). After attaching the liner (11) to the application element (10) the aluminum foil sticks to the cooling plate (40) for promoting the heat transfer from the cooling plate via the aluminum foil to the injection site of the patient who is receiving the injection. FIG. 8B shows a further example of a suitable liner. It differs from the liner shown in FIG. 8A in that only a section of the distal part of the liner is composed of a single-sided adhesive aluminum foil. The remaining part of the distal part of the liner is composed of plastic. The section composed of the aluminum foil is the section of the liner which contacts the cooling plate (40) of the injection device after it is attached to the application element (10). The shown injection device allows 8 to 10 injections into distinct injection sites, wherein each upcoming injection site is cooled down to a sufficient low temperature below 8° C., preferably to a temperature of about 1° C. to about 4° C., while the injection is performed in the previous cooled injection site.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The following are some exemplary numbered embodiments of the present disclosure.
1. An injection device (100) for introducing a substance into a subject, comprising: (a) an application element (10); (b) a housing (50) having a receptacle for holding a syringe; (c) an electromechanical drive device system arranged to advance a piston for the syringe, and (d) a cooling assembly comprising a cooling plate (40), wherein the cooling plate (40) is incorporated into the application element (10). Within various embodiments of the above the cooling plate (40) is configured to cool down an upcoming injection site by contacting said injection site.
Within other embodiments an injection device for introducing a substance into a subject is provided comprising a housing, an application element, a drive system, and a cooling assembly comprising a Peltier element, wherein the Peltier element is adjacent to, but does not surround two, three, or four sides of an injection site. Within related embodiments the injection device further comprises a needle housing comprising one or more needles.
Within other embodiments the injection device has a drive system for advancing a piston of the syringe and a drive system for advancing the syringe itself. In one embodiment both drive systems may be advanced simultaneously, but the syringe may be retracted separately at a faster rate than the piston (e.g., the syringe may be retracted and the piston remain static, thereby injecting fluid into the subject). Within other embodiments the syringe and piston may be advanced simultaneously, followed by further advancement of the syringe (thereby injecting fluid into the subject). Within yet further embodiments the drive systems may be adjusted, for example, by the user or by a set protocol (e.g., to account for different viscosities of an injected substance).
2. The injection device (100) according to embodiment 1, wherein the cooling assembly comprises a cooling shoe (45), a cooling element, in particular a Peltier element (41), a heat pipe (42), a heat exchanger (43) and at least one fan (44).
3. The injection device (100) according to embodiment 1 or 2, wherein the cooling plate (40) is part of the cooling shoe (45) and the cooling shoe is in contact with the cooling element, in particular the Peltier element (41).
4. The injection device (100) according to embodiments 1, 2, and/or 3, wherein the distal surface of the cooling plate has the form of a single area, preferably of a rectangle, a square, a circular area, or an oval area.
5. The injection device according to embodiments 1, 2, 3, and/or 4, wherein the cooling power per area of the cooling plate (40) is about 1.5 W/cm² to about 10 W/cm², more preferably about 2 W/cm² to about 10 W/cm². Within further embodiments, the cooling power cycles between at least two different power settings.
6. The injection device (100) according to embodiments 2, 3, 4, and/or 5, wherein the cooling shoe is floating in said housing. Within various embodiments, the cooling shoe can be mounted at a single position in the housing (e.g. on the ribs, fins or protruding portions of the housing, and/or being fixed by a tongue-and-groove joint (49, 55) in the housing).
7. The injection device (100) according to embodiments 1, 2, 3, 4, 5, and/or 6 wherein said housing (50) has intake vents (51) and outflow vents (52). Within certain embodiments of the invention the intake vents may be laterally arranged on the housing. Within other embodiments the housing (50) has outflow vents (52), which may be arranged at the back and/or the bottom of the proximal part of the injection device.
8. The injection device (100) according to embodiments 1, 2, 3, 4, 5, 6 and/or 7, wherein the injection device comprises one or more non-mechanical sensors such as an optical sensor and/or a magnetic sensor.
9. The injection device (100) according to embodiments 1, 2, 3, 4, 5, 6, 7, and/or 8 further comprising a temperature sensor. Within yet other embodiments the injection device may further comprise a light source, suitable (for example), for illuminating an injection area.
10. The injection device (100) according to any one of embodiments 1, 2, 3 4, 5, 6, 7, 8 and/or 9 further comprising a needle housing (21) having one or more needles.
11. The injection device according to embodiment 10 wherein said injection device or part thereof is configured to be activated only if a needle housing (21) is coupled to the injection device.
12. A needle housing configured for fitting into the injection device according to any one of embodiments 1, 2, 3, 4, 5, 6, 7, 8, 9, 10, 11 and/or 12, wherein the needle housing is locked in the injection device after its insertion. Within certain embodiments, insertion of the needle housing in the injection device is required for activating the injection device or a part thereof.
13. The injection device according to any embodiments 1, 2, 3, 4, 5, 6, 7, 8 9, 10 and/or 11, and the needle housing of embodiment 12, wherein the application element is configured to be coupled to a liner (11).
14. The injection device (100) according to any one of embodiments 1, 2, 3, 4, 5, 6, 7, 8, 9, 10, 11, and/or 13, further comprising a communication interface. Within further embodiments, the injection device may further comprise a control unit, which activates and controls the injection device. Representative examples of a control unit are disclosed in WO 2016/022865, which is incorporated by reference in its entirety. Within related embodiments the injection device may be powered by direct current, a battery, or current supplied from a control unit.
15. A liner (11) configured to be coupled to the application element of the injection device according to any embodiments 1, 2, 3, 4, 5, 6, 7, 8 9, 10, 11, 13 and/or 14 and the needle housing of embodiment 12, wherein the liner comprises a heat-conducting material (12), part or item which contacts the distal surface of the cooling plate (40) after its coupling to the application element (10).
16. A kit, comprising a needle housing having one or more needles, a liner, and a syringe. Within related embodiments, the kit may also comprise an injection device according to any one of embodiments 1, 2, 3, 4, 5, 6, 7, 8, 9, 10, 11, 13 and/or 14.
17. The kit according to embodiment 16 further comprising an injectable composition. Representative injectable compositions include both liquids and suspensions, examples of which include pharmaceutical agents, biologic agents, cosmetic agent, and cells.
18. A method for introducing a substance into the tissue of a subject comprising:
a) selecting a first exterior surface of skin of a subject as a first injection site;
b) placing the cooling plate of an injection device according to any one of embodiments 1, 2, 3, 4, 5, 6, 7, 8, 9, 10, 11, 13, and/or 14 on the first injection site and cooling it to a desired temperature;
c) moving the injection device in a position allowing an injection into the previously cooled injection site;
d) penetrating the first surface with a needle or simultaneously with all needles of a needle array of the injection device, wherein all needles of the needle array extending from the first surface by a uniform distance into the subject;
e) ejecting a substance out of the needle/s and into the tissue of the patient; and
f) withdrawing the one needle or simultaneously all needles of the needle array from the subject;
g) optionally, simultaneously to step d), e) and/or f) cooling the upcoming injection site to a desired temperature; and
h) optionally, repeating steps c) to f) and optionally g).

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. An injection device (100) for introducing a substance into a subject, comprising: (a) an application element (10); (b) a housing (50) having a receptacle for holding a syringe; (c) an electromechanical drive device system arranged to advance a piston for the syringe, and (d) a cooling assembly comprising a cooling plate (40), wherein the cooling plate (40) is incorporated into the application element (10).

2. The injection device (100) according to claim 1, wherein the cooling assembly comprises a cooling shoe (45), a cooling element, in particular a Peltier element (41), a heat pipe (42), a heat exchanger (43) and at least one fan (44).

3. The injection device (100) according to any one of the proceeding claims, wherein the cooling plate (40) is part of the cooling shoe (45) and the cooling shoe is in contact with the cooling element, in particular the Peltier element (41).

4. The injection device (100) according to any one of the proceeding claims, wherein the distal surface of the cooling plate has the form of a single area, preferably of a rectangle, a square, a circular area, or an oval area.

5. The injection device (100) according to claim 2 or 3, wherein the cooling shoe is floating in said housing.

6. The injection device (100) according to any one of the proceeding claims, wherein said housing (50) has intake vents (51) and outflow vents (52).

7. The injection device (100) according to any one of the proceeding claims further comprising a temperature sensor.

8. The injection device (100) according to any one of the proceeding claims further comprising a needle housing (21) having one or more needles.

9. The injection device according to claim 8 wherein said injection device or part thereof is configured to be activated only if a needle housing (21) is coupled to the injection device.

10. A needle housing configured for fitting into the injection device according to any one of claims 1 to 9, wherein the needle housing is locked in the injection device after its insertion.

11. The injection device according to any one of claims 1 to 10, wherein the application element is configured to be coupled to a liner (11).

12. The injection device (100) according to any one of the proceeding claims, further comprising a communication interface.

13. A liner (11) configured to be coupled to the application element of the injection device according to any one of the preceeding claims, wherein the liner comprises a heat-conducting material (12), part or item which contacts the distal surface of the cooling plate (40) after its coupling to the application element (10).

14. A kit, comprising a needle housing having one or more needles, one or more liners, and a syringe.

15. The kit according to claim 16 further comprising an injectable composition.

16. A method for introducing a substance into the tissue of a subject comprising:
a) selecting a first exterior surface of skin of a subject as a first injection site;
b) placing the cooling plate of an injection device according to any one of claims 1 to 9 on the first injection site and cooling it to a desired temperature;
c) moving the injection device in a position allowing an injection into the previously cooled injection site;
d) penetrating the first surface with one needle or simultaneously with all needles of a needle array of the injection device, wherein all needles of the needle array extending from the first surface by a uniform distance into the subject;
e) ejecting a substance out of the needle/s and into the tissue of the patient; and
f) withdrawing the one needle or simultaneously all needles of the needle array from the subject;
g) optionally, simultaneously to step d), e) and/or f) cooling the upcoming injection site to a desired temperature; and
h) optionally, repeating steps c) to f) and optionally g).
